(19) United States
(12) Patent Application Publication
(10) Pub. No.: US 2009/0171078 A1
(43) Pub. Date: Jul. 2, 2009

(54) METHOD OF SEQUENCING NUCLEIC ACIDS USING ELABORATED NUCLEOTIDE PHOSPHOROTIOLATE COMPOUNDS

(75) Inventors: Kai Qin LAO, Pleasanton, CA (US); Neil A. Straus, Emeryville, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applied Biosystems Inc., Foster City, CA (US)

(21) Appl. No.: 12/275,176

(22) Filed: Nov. 20, 2008

Related U.S. Application Data
(60) Provisional application No. 61/004,038, filed on Nov. 20, 2007.

(51) Int. Cl. C07H 19/04; C12P 19/34; C12Q 1/68

(52) U.S. Cl. 536/26.1; 435/91.2; 435/6

(57) ABSTRACT

The present teachings provide methods, compositions, and kits for synthesizing and sequencing nucleic acids. In some embodiments, elaborated nucleotide phosphorothiolate compounds are employed along with efficient cleaving reactions. Improved sequencing efficiency is achieved by the rapid polymerase-mediated incorporation of elaborated nucleotide phosphorothiolate compounds. Increased sequencing efficiency is also achieved by the ability of the cleaving reactions to restore the incorporated nucleotides to their natural structure prior to subsequent elongation.

### CROSS-REFERENCE TO RELATED APPLICATIONS

This application claims a priority benefit under 35 U.S.C. § 119(e) from U.S. Patent Application No. 61/004,038, filed Nov. 20, 2007, which is incorporated herein by reference.

### FIELD

The present teachings generally relate to methods for synthesizing and sequencing nucleic acids.

### BACKGROUND

The detection of the presence or absence of (or quantity of) one or more target nucleic acids in a sample or samples containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

The gold standard in nucleic acid sequencing is capillary electrophoresis employing labeled dideoxy-nucleotides. Recently, next generation sequencing approaches have been described, bearing the promise of increased speed, throughput, and accuracy, and lower cost. Certain of these approaches employ polymerase-mediated incorporation of reversible terminator compounds (see for example U.S. Pat. No. 6,664,079). Other next-generation sequencing approaches employ ligation-mediated strategies (see for example WO2006/084132). Trade-offs in speed, accuracy, and cost continue to plague next generation sequencing approaches. The present teachings combine the strengths of polymerase-mediated approaches with certain aspects of ligation-mediated approaches to provide improved methods of performing highly parallel next generation sequencing.

### SUMMARY

A method of synthesizing a nucleic acid comprising;

(a) hybridizing a primer to a template;

(b) polymerase extending the primer with an elaborated mono-nucleotide phosphorothiolate compound to form an extension product, wherein the elaborated mono-nucleotide phosphorothiolate compound comprises a 3′C—O—PO₂—S—X group;

(c) cleaving the 3′C—O—PO₂—S—X group with a phosphorothiolate cleaving agent, wherein the S—X are removed from the extension product, to leave a 3′PO₄ group;

(d) hydrolyzing the 3′PO₄ group with a phosphate removing agent to leave a 3′OH; and

(e) repeating (b)-(d) to synthesize the nucleic acid.

Methods of sequencing are also provided, as are kits and compositions.

### BRIEF DESCRIPTION OF THE FIGURES

FIG. 1 shows an illustrative embodiment according to the present teachings.

FIG. 2 shows an illustrative embodiment according to the present teachings.

FIG. 3 shows an illustrative embodiment according to the present teachings.

FIG. 4 shows an illustrative embodiment according to the present teachings.

FIG. 5 shows an illustrative embodiment according to the present teachings.

### DETAILED DESCRIPTION OF CERTAIN EMBODIMENTS

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in this application, this application controls.

The use of the singular includes the plural unless specifically stated otherwise. The word “a” or “an” means “at least one” unless specifically stated otherwise. The use of “or” means “and/or” unless stated otherwise. The use of “or” in the context of multiply dependent claims means the alternative only. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise. All ranges discussed herein include the endpoints and all values between the endpoints.

### DEFINITIONS

As used herein, the term “nucleotide” includes native (naturally occurring) nucleotides, which include a nitrogenous base selected from the group consisting of adenine, thymidine, cytosine, guanine and uracil, a sugar selected from the group of ribose, arabinose, xylose, and pyranose, and deoxyribose (the combination of the base and sugar generally referred to as a “nucleoside”), and one to three phosphate groups, and which can form phosphodiester internucleosidyl linkages. Further, as used herein “nucleotide” refers to nucleotide analogs. Such analogs can have a sugar analog, a base analog and/or an internucleosidyl linkage analog. Additionally, analogs exhibiting non-standard base pairing are also included (see for example U.S. Pat. No. 5,432,272). Such nucleotide analogs include nucleotides that are chemically modified in the natural base (“base analogs”), chemically modified in the natural sugar (“sugar analogs”), and/or chemically modified in the natural phosphodiester or any other internucleosidyl linkage (“internucleosidyl linkage analogs”). In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose,2′-bromoribose, 2′iodoribose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a nucleic acid include, but are not limited to, the structures:

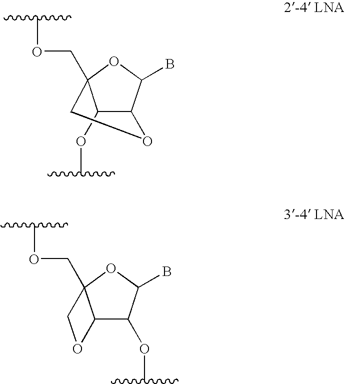

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

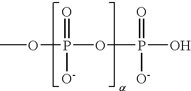

where α is an integer from 0 to 4. In certain embodiments, α is 2 and the phosphate ester is attached to the 3′- or 5′carbon of the pentose. In certain embodiments, the nucleotides are those in which the nucleotide base is a purine, a 7-deazapurine, a pyrimidine, a pyrazolopyrimidine, or an analog thereof of the aforementioned. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

In certain embodiments, exemplary phosphate ester analogs include, but are not limited to, alkylphosphonates, methylphosphonates, phosphoramidates, phosphotriesters, phosphorothiolates, phosphorodithiolates, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into nucleic acid analogs in which the DNA/RNA phosphate ester and/or sugar phosphate ester backbone is replaced with a different type of internucleotide linkage. Exemplary nucleic acid analogs include, but are not limited to, DNA with one or more phosphorothiolates in one or both of its backbones, and peptide nucleic acids.

As used herein, the term “universal nucleotide”, “universal nucleoside”, and “universal base”, refer to compounds that exhibit the ability to incorporate into extension products, and which can form base pair with more than one conventional nucleotide. One example of a universal base is inosine. Illustrative universal nucleotides, nucleosides, and bases, can be found in U.S. Pat. No. 7,169,557, U.S. Pat. No. 7,214,783, Published PCT WO 01/72764A1, and Seela et al., N.A.R. 2000, Vol 28, No. 17, 3224-3232.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, K⁺, and the like. A nucleic acid may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A nucleic acid may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

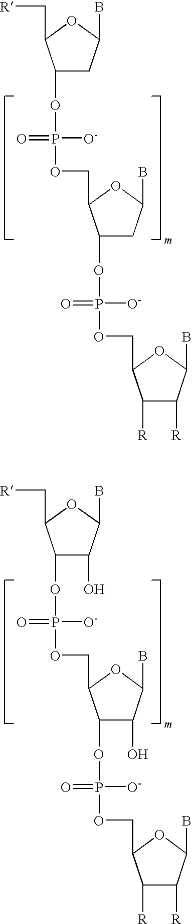

wherein each B is independently the base moiety of a nucleotide, e.g., a purine, a 7-deazapurine, a pyrimidine, a pyrazolopyrimidine, or an analog thereof of the aforementioned. Each m defines the length of the respective nucleic acid and can range from zero to thousands, tens of thousands, or even more; each R is independently selected from the group comprising hydrogen, hydroxyl, halogen, —R″, —OR″, and —NR″R″, where each R″ is independently (C₁-C₆)alkyl or (C₅-C1₄)aryl, or two adjacent Rs may be taken together to form a bond such that the ribose sugar is 2′,3′-didehydroribose, and each R′ may be independently hydroxyl or

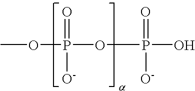

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A nucleic acid analog may comprise one or more lesions. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; (v) phosphorodithioate; (vi) phosphorothiolate and (vii) phosphorodithiolate.

The terms “annealing” and “hybridization” are used interchangeably and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, templates, polynucleotides, oligonucleotides, and primers.

The term “primer” or “oligonucleotide primer” as used herein, refers to an oligonucleotide from which a primer extension product can be synthesized under suitable conditions. In certain embodiments, such suitable conditions comprise the primer being hybridized to a complementary nucleic acid and incubated in the presence of, for example, nucleotides, a polymerization-inducing agent, such as a DNA or RNA polymerase, at suitable temperature, pH, metal concentration, salt concentration, etc. In various embodiments, primers are 5 to 100 nucleotides long. In various embodiments, primers are 8 to 75, 10 to 60, 10 to 50, 10 to 40, or 10 to 35 nucleotides long.

The term “target nucleic acid” as used herein refers to an RNA or DNA that has been selected for detection. Exemplary RNAs include, but are not limited to, mRNAs, tRNAs, snRNAs, rRNAs, retroviruses, small non-coding RNAs, microRNAs, polysomal RNAs, pre-mRNAs, intronic RNA, and viral RNA. Exemplary DNAs include, but are not limited to, genomic DNA, plasmid DNA, phage DNA, nucleolar DNA, mitochondrial DNA, chloroplast DNA, cDNA, synthetic DNA, yeast artificial chromosomal DNA (“YAC”), bacterial artificial chromosome DNA (“BAC”), other extrachromosomal DNA, and primer extension products. Generally, the templates to be sequenced in the present teachings are derived from any of a variety of such target nucleic acids, themselves derived from any of a variety of samples.

The term “sample” as used herein refers to any sample that is suspected of containing a target analyte and/or a target nucleic acid. Exemplary samples include, but are not limited to, prokaryotic cells, eukaryotic cells, tissue samples, viral particles, bacteriophage, infectious particles, pathogens, fungi, food samples, bodily fluids (including, but not limited to, mucus, blood, plasma, serum, urine, saliva, and semen), water samples, and filtrates from, e.g., water and air.

As used herein, the term “amplification” refers to any method for increasing the amount of a target nucleic acid, or amount of signal indicative of the presence of a target nucleic acid. Illustrative methods include the polymerase chain reaction (PCR), rolling circle amplification (RCA), helicase dependant amplification (HDA), Nucleic Acid Sequence Based Amplification (NASBA), ramification amplification method (RAM), recombinase-polymerase amplification (RPA), multiple strand displacement amplification (MDA), and others. In some embodiments of the present teachings, amplification can occur in an emulsion PCR, containing primer-immobilized microparticles, as described for example in WO2006/084132, which is hereby incorporated by reference in its entirety for any purpose.

As used herein, the term “label” refers to detectable moieties that can be attached to nucleotides directly or indirectly to thereby render the molecule detectable by an instrument or method. For example, a label can be any moiety that: (i) provides a detectable signal; (ii) interacts with a second label to modify the detectable signal provided by the first or second label; or (iii) confers a capture function, e.g. hydrophobic affinity, antibody/antigen, ionic complexation. The skilled artisan will appreciate that many different species of labels can be used in the present teachings, either individually or in combination with one or more different labels. Exemplary labels include, but are not limited to, fluorophores, radioisotopes, Quantum Dots, chromogens, Sybr Green™, enzymes, antigens including but not limited to epitope tags, heavy metals, dyes, phosphorescence groups, chemiluminescent groups, electrochemical detection moieties, affinity tags, binding proteins, phosphors, rare earth chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal)ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal)ruthenium (II), also known as Ru(v-bpy₃²⁺), and the like.

As used herein, the term “fluorophore” refers to a label that comprises a resonance-delocalized system or aromatic ring system that absorbs light at a first wavelength and emits fluorescent light at a second wavelength in response to the absorption event. A wide variety of such dye molecules are known in the art, as described for example in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Exemplary fluorescein-type parent xanthene rings include, but are not limited to, the xanthene rings of the fluorescein dyes described in U.S. Pat. Nos. 4,439,356, 4,481,136, 4,933,471 (Lee), 5,066,580 (Lee), 5,188,934, 5,654,442, and 5,840,999, WO 99/16832, EP 050684, and U.S. Pat. Nos. 5,750,409 and 5,066,580. Additional rhodamine dyes can be found, for example, in U.S. Pat. Nos. 5,366,860 (Bergot et al.), 5,847,162 (Lee et al.), 6,017,712 (Lee et al.), 6,025,505 (Lee et al.), 6,080,852 (Lee et al.), 5,936,087 (Benson et al.), 6,111,116 (Benson et al.), 6,051,719 (Benson et al.), 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. Additional typical fluorescein dyes can be found, for example, in U.S. Pat. Nos. 5,750,409, 5,066,580, 4,439,356, 4,481,136, 4,933,471 (Lee), 5,066,580 (Lee), 5,188,934 (Menchen et al.), 5,654,442 (Menchen et al.), 6,008,379 (Benson et al.), and 5,840,999, PCT publication WO 99/16832, and EPO Publication 050684. In some embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

The labels of the present teachings can be attached to any suitable position of the elaborated nucleotide phosphorothiolate compounds of the present teachings. For example, illustrative teachings regarding attaching a label to the base of a nucleotide can be found in U.S. Pat. No. 6,664,079, which is hereby incorporated by reference in its entirety for any purpose.

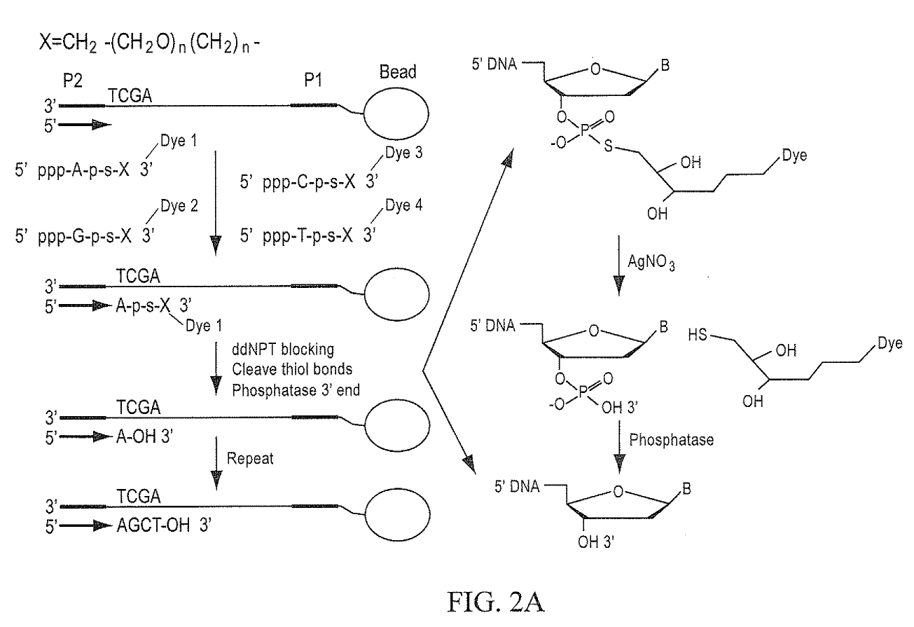

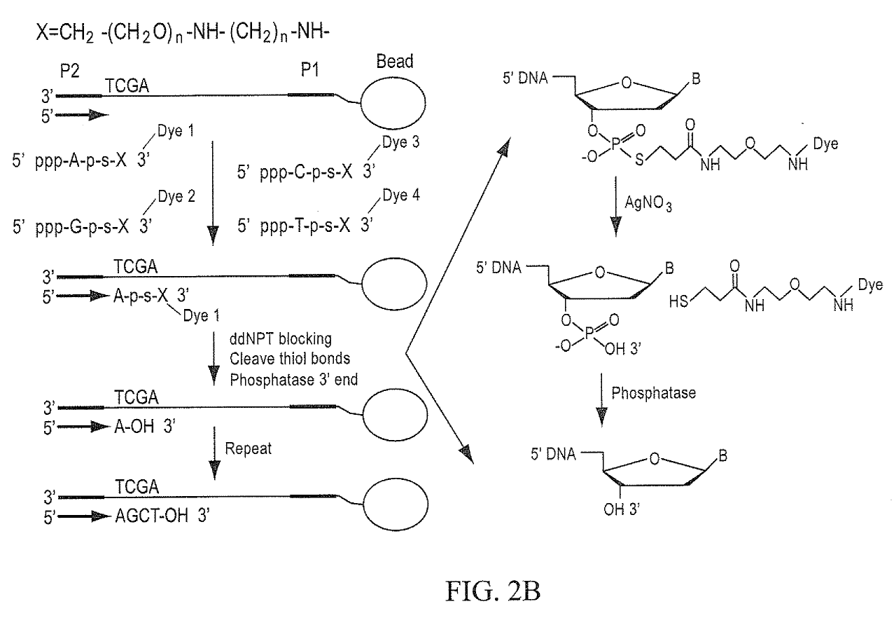

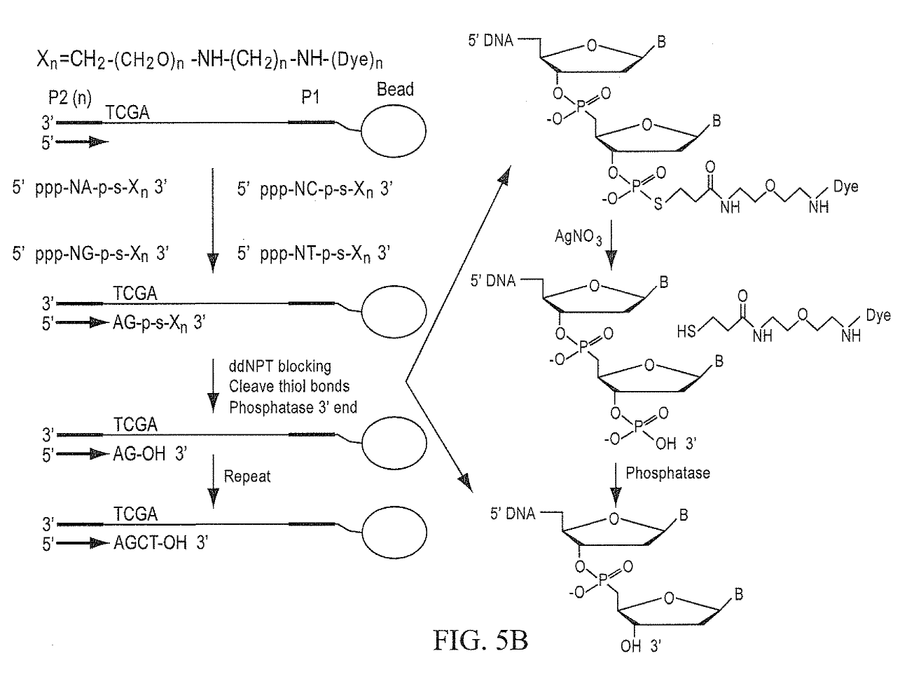

The term “blocking moiety” refers to any structural feature comprising the X of the 3′C—O—PO₂—S—X group of the erstwhile terminal nucleotide, which prevents the subsequent addition of nucleotides into a growing extension product. Such blocking can result from the absence of a hydroxyl group at the appropriate position, such as the 3′ carbon position when the X itself comprises a nucleotide, such as a di-deoxynucleotide. Any of a variety of blocking moieties can be employed. In some embodiments, the blocking moieties are chosen to structurally resemble nucleotides or parts of the nucleotide, thus taking advantage of the ability of certain polymerase to incorporate di-nucleotides (e.g. U.S. Pat. No. 7,060,440). Such blocking groups include carbohydrates such as substituted thiol glycerol. The substituted thiol glycerol can further be elaborated with a label such as a fluorophore. Additional blocking moieties include carbamates, ethers, glycerol, a —(CH₂)ₐCO—NH—(CH2)_{b}—O—(CH2)_{c}—NH (as shown for example, in FIGS. 2B and 5B, where for example a=0-10, b=0-10, and c=0-10)) any carbohydrate including four-carbon carbohydrates, three-carbon carbohydrates, and two-carbon carbohydrates, a dideoxynucleotide, a dideoxynunleotide containing a universal base, CH₂(CH₂O)n(CH2)m, an ether group, an ester group, a substituted carbohydrate, a carbamate, or a phosphoamidite. In some embodiments, the CH₂(CH₂O)n(CH2)m can comprise n=0-8, in some embodiments n=0-5, in some embodiments, n=1-5. In some embodiments, the CH₂(CH₂O)n(CH2)m can comprise m=0-30, in some embodiments m=0-15, in some embodiments m=0-10, in some embodiments, m=1-5. Generally, various blocking moieties are known in the art, and can be found for example in U.S. Pat. No. 6,664,079, U.S. Pat. No. 5,763,594, PCT Publication WO9106678, PCT Publication WO0053805, PCT Publication WO0050642, PCT Publication WO09305183, PCT Publication WO09735033, U.S. Pat. No. 6,232,465, U.S. Pat. No. 6,632,655, U.S. Pat. No. 6,087,095, U.S. Pat. No. 5,908,755, U.S. Pat. No. 5,302,509, all of which are hereby expressly incorporated by reference in their entirety for any purpose.

As used herein, the term “first nucleotide” refers to the upstream most nucleotide of an elaborated mono-nucleotide phosphorothiolate compound, elaborated di-nucleotide phosphorothiolate compound, or elaborated oligonucleotide phosphorothiolate compound. First nucleotides are also generally also known in the art as the 5′-most nucleotide. The nucleotide following the “first nucleotide” is referred to herein as a “second nucleotide.”

As used herein, the term “shifted primer” refers to a primer which, relative to the position on a template at which another primer hybridizes, is shifted an appropriate number of nucleotides to allow for sequence decoding according to the present teachings. In some embodiments, the shift is an odd number of nucleotides. Typically in an embodiment in which elaborated di-nucleotides are incorporated and two nucleotides remain in the extension product following the cleavage reactions, the shifted primer will be shifted one nucleotide relative to the other primer, but shifts of any odd number of nucleotides are contemplated by the present teachings, including three, five, seven, etc. Such shifts can be shown as “n−1” in certain of the figures. It will be appreciated that the shift can be upstream or downstream relative to the position of the earlier primer.

As used herein, the term “suitable polymerase” refers to any polymerase that incorporates the desired elaborated nucleotide phosphorothiol compounds of the present teachings into an extension product. Included are DNA-dependent DNA polymerases, RNA-dependent DNA polymerases, DNA-dependent RNA polymerases, and RNA-dependent RNA polymerases. Illustrative examples can be found, for example in U.S. Pat. No. 7,060,440, which is hereby incorporated by reference in its entirety for any purpose, and include the 543 amino acids of the C-terminus of Taq polymerase, Klenow (Exo-) DNA polymerase (commercially available from Fermentas), AMPLITAQ (commercially available from Applied Biosystems) and Tth DNA polymerase (commercially available from Promega). Other polymerases can be used, as routine experimentation will provide.

As used herein, the term “phosphorothiolate cleaving agent” refers to the use of any suitable substance that can cleave the phosphorothiolate group between the terminal nucleotide and downstream blocking moieties. In other words, the phosphorothiolate cleaving agent can cleave the —SX from 3′C—O—PO₂—S—X. For example, AgNO3 can be employed, as well as any of a variety of transition metals, any of a variety of salts of transition metals. For example, the metal can be Au, Ag, Hg, Cu, Mn, Zn, or Cd. The agent can be a water-soluble salt that provides Au+, Ag+, Hg+, Cu+, Mn,+, Zn+, or Cd+ anions. Salts that provide ions of other oxidation states can also be used. In some embodiments, silver-containing salts such as silver nitrate (AgNO3), or other salts that provide Ag+ ions are used. Suitable conditions include, for example, 50 mM AgNO3 at about 22-37 C for 10 minutes or more, for example 30 minutes. Preferably the pH is between 4.0 and 10.0, more preferably between 5.0 and 9.0, e.g., between 6.0-8.0. In some embodiments, the pH is about 7. Further discussion can be found in Mag et al., Nucleic Acids Research, 19(7): 1437-1441, 1991. In some embodiments, Iodine, can be employed, as described for example in Vyle et al., Biochemistry, 1992, 3012-3018. Also, other halogens can be employed. Generally, this cleavage will result in a phosphate group on the 3′ carbon of the terminal nucleotide.

As used herein, the term “phosphate removing agents” refers to the use of any suitable substance that can restore a free —OH group to the 3′ carbon of the terminal nucleotide, thus resulting in the generation of an extendable terminus. For example, a phosphatase can be employed such as alkaline phosphatase, as well as any of a variety of enzymes such as T4 polynucleotide kinase commercially available from NEB, and other substances. This restoration typically involves the direct removal of a PO₄ group to leave an OH group.

As used herein, the term “terminal nucleotide” refers to the extendable nucleotide that remains hybridized to the template and incorporated into the extension product, following the restoration of the OH group on the 3′ carbon from the PO₄ group. In some embodiments, elaborated mono-nucleotides can be employed that contain a single nucleotide with a blocking moiety. In such embodiments, the nucleotide that remains hybridized to the template and incorporated into the extension product following treating with a phosphorothiolate cleaving agent, and phosphate removing agent, is the “terminal nucleotide.” In some embodiments, elaborated di-nucleotides can be employed that contain a first nucleotide and a second nucleotide, wherein the second nucleotide contains the blocking moiety downstream of the second nucleotide's 3′ carbon. In such embodiments, the second of the two nucleotides that remains hybridized to the template and incorporated into the extension product following treating with a phosphorothiolate cleaving agent, and phosphate removing agent, is the “terminal nucleotide.”

As used herein, the term “elaborated nucleotide phosphorothiolate compound” refers to any of a variety of elaborated mono-nucleotide phosphorothiolate compounds, elaborated di-nucleotide phosphorothiolate compounds, and elaborated oligonucleotide phosphorothiolate compounds, all of which contain a phosphorothiolate group in the form of a 3′C—O—PO₂—S—X, and all of which contain a trisphosphate at the 5′ carbon of the first nucleotide. The organic synthesis of phosphorothiolate containing nucleotides has been described, and can be found for example in Kresse et al., N.A.R., Volume 2, Number 1, January 1975; Vyle et al., Biochemistry, 1992, 31, 3012-3018; Cosstick et al., N.A.R. Vol 18, No. 4, 1990; Rybakov et al., N.A.R. Vol 9, Number 1, 1981. An “elaborated mono-nucleotide phosphorothiolate compound” contains a first nucleotide, and this first nucleotide contains a 3′C—O—PO₂—S—X group. Such “elaborated mono-nucleotide phosphorothiolate compounds are also referred to herein as simply “elaborated mono-nucleotides.” An “elaborated di-nucleotide phosphorothiolate compound” contains a first nucleotide and a second nucleotide, and the second nucleotide contains a 3′C—O—PO₂—S—X group. Such “elaborated di-nucleotide phosphorothiolate compounds” are also referred to as simply “elaborated di-nucleotides.”The synthesis and use of various di-nucleotide compounds is described in U.S. Pat. No. 7,060,440. An “elaborated oligonucleotide phosphorothiolate compound” contains a first nucleotide, a second nucleotide, and one or more additional nucleotides, and the 3′ most nucleotide of the one or more additional nucleotides contains a 3′C—O—PO₂—S—X group. Such “elaborated oligonucleotide phosphorothiolate compounds” are also referred to as simply “elaborated oligonucleotides.”

Put in other words, the terms “mono”, “di”, and “oligo”, in the context of the elaborated nucleotide phosphorothiolate compounds of the present teachings, refer to the number of nucleotides that remain incorporated in the extension product following the cleavage of the phosphorothiolate group and the hydrolysis of the phosphate group. As a clarifying example, an elaborated di-nucleotide of the present teachings can actually contain three nucleotides if the third nucleotide is a non-extendable nucleotide (such as a dideoxy-nucleotide) comprising the X of the 3′C—O—PO₂—S—X group. Such a compound is referred to as an elaborated di-nucleotide, and not an elaborated oligonucleotide, because two nucleotides will remain incorporated in the extension product following the cleavage of the phosphorothiolate group and the hydrolysis of the phosphate group.

### Certain Exemplary Methods

Methods provided herein may be carried out in any order of the recited events that is logically possible, as well as the recited order of events. Standard techniques may be used for recombinant DNA and oligonucleotide synthesis. Enzymatic reactions and purification techniques may be performed according to manufacturer's specifications and/or as commonly accomplished in the art and/or as described herein. The foregoing techniques and procedures may be generally performed according to conventional methods known in the art and as described in various general and more specific references, including but not limited to, those that are cited and discussed throughout the present specification. See, e.g., Sambrook et al. Molecular Cloning: A Laboratory Manual (2d ed., Cold Spring Harbor Laboratory Press, Cold Spring Harbor, N.Y. (1989)); Lehninger, Biochemistry (Worth Publishers, Inc.); Methods in Enzymology (S. Colowick and N. Kaplan Eds., Academic Press, Inc.); Oligonucleotide Synthesis (N. Gait, ed., 1984); A Practical Guide to Molecular Cloning (2^{nd} Ed., Wily Press, 1988). Unless specific definitions are provided, the nomenclatures utilized in connection with, and the laboratory procedures and techniques of, biology, biochemistry, analytical chemistry, and synthetic organic chemistry described herein are those known and used in the art.

### Sequencing With Elaborated Mono-Nucleotide-Phosphorothiolate Compounds, Wherein X Contains a Nucleotide

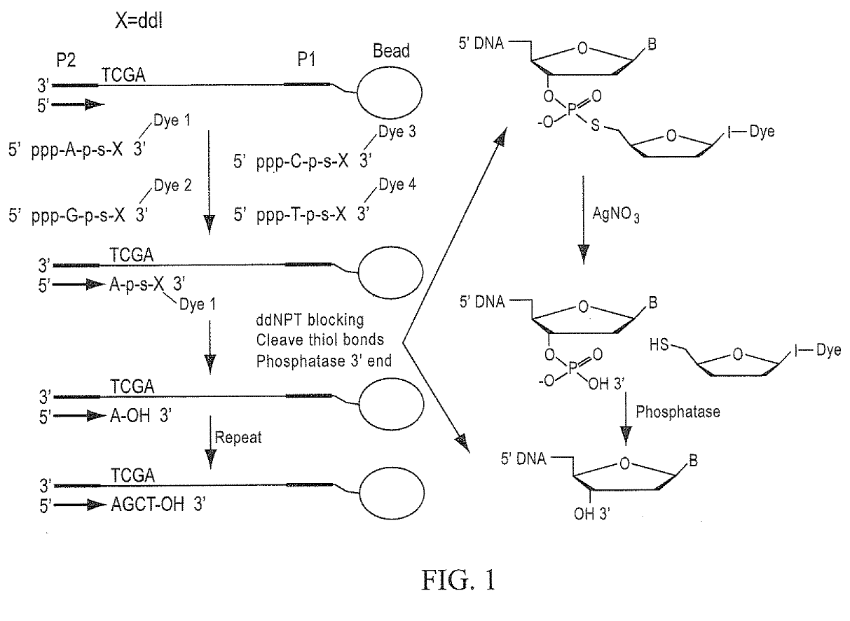

A first aspect of the present teachings is provided in FIG. 1. Here, a template polynucleotide is shown attached to a bead. The template can have known ends that can be queried by primers, for example a “P1” end proximal to the bead and a “P2” end distal to the bead. A primer complementary to the P2 end can be hybridized to the template, thus forming a substrate suitable for polymerization with a suitable polymerase. For illustration, the template is shown containing a sequence TCGA from the 3′ to 5′ direction.

Proceeding with this example, a group of four elaborated mono-nucleotides can be presented. This group of four elaborated mono-nucleotides can contain a different first nucleotide, but contain a dideoxy-inosine as the X moiety of the 3′C—O—PO₂—S—X. The di-deoxyinosine of each of the four elaborated mono-nucleotides serves as a blocking moiety, such that incorporation of a given elaborated mono-nucleotide into an extension product halts incorporation of additional elaborated mono-nucleotides. Each of the four elaborated mono-nucleotides in the group can contain a distinct label on the inosine, for example the label can be attached to the base of the inosine. These labels are shown as Dye 1, Dye 2, Dye 3, and Dye 4. Detection of Dye 1 indicates the incorporation of the elaborated mono-nucleotide containing 5′-ppp-A-p-S-ddI-Dye-1-3′. Thus, the first nucleotide sequenced in the template is a T.

In FIG. 1, a phosphorothiolate group (note the Sulfur, s) is present between the first nucleotide and the di-deoxy inosine. This phosphorothiolate group (“thiol bonds”) can now be cleaved with a phosphorothiolate cleaving agent, for example a metal compound such as AgN0₃. This cleavage allows for the removal of the inosine group, and its associated Dye and di-deoxy blocking moiety. This cleavage will result in a phosphate group on the 3′ carbon of the first nucleotide. Treating with a phosphate removing agent, using for example a phosphatase, can restore a free —OH group to the 3′ carbon of the first nucleotide, resulting in the generation of a terminal nucleotide. A next round of elaborated mono-nucleotide incorporation can then be performed and the process repeated successively to determine the sequence of the template.

### Sequencing by Elaborated Mono-Nucleotide Phosphorothiolate Compounds

A second aspect of the present teachings is presented in FIG. 2A. Here, a template polynucleotide is shown attached to a bead. The template can have known ends that can be queried by primers, for example a “P1 ” end proximal to the bead and a “P2” end distal to the bead. A primer complementary to the P2 end can be hybridized to the template, thus forming a substrate suitable for polymerization with a suitable polymerase. For illustration, the template is shown containing a sequence TCGA from the 3′ to 5′ direction.

Proceeding with this example, a group of four elaborated mono-nucleotide phosphorothiolate compounds can be presented. This group of four elaborated mono-nucleotide phosphorothiolate compounds can contain different first nucleotides. Each elaborated mono-nucleotide phosphorothiolate compound contains a blocking moiety attached to the 3′ carbon, such as a —CH₂—(CH₂O)n(CH₂)n moiety, depicted as X. Without intending to be limited by any particular theoretical basis, blocking moieties can be chosen that are predicted to be sufficiently structurally similar to the second nucleotide of a di-nucleotide so as to allow for polymerase-mediated incorporation into an extension product, taking advantage of the demonstrated ability of certain polymerases to incorporate di-nucleotides into growing extension products (see U.S. Pat. No. 7,060,440). Thus, incorporation of a given elaborated mono-nucleotide into an extension product prevents the addition of subsequent elaborated mono-nucleotides. Further, each of the four elaborated mono-nucleotide phosphorothiolate compounds in the group can contain a distinct Dye on the blocking moiety. Incorporation of an elaborated mono-nucleotide phosphorothiolate compound, and subsequent detection of Dye 1, indicates the incorporation 5′-ppp-A-p-S-blocking-moiety-Dye-1. Thus, the first nucleotide sequenced in the template is a T.

The phosphorothiolate group present between the first nucleotide and the blocking moiety can now be cleaved with a phosphorothiolate cleaving agent, allowing for the removal of the blocking moiety, and its associated Dye. Cleavage can be employed any number of ways, as discussed supra, and is shown here being performed with AgNO3. This cleavage will result in a phosphate group on the 3′ carbon of the first nucleotide. Treating with a phosphate removing agent, shown in the Figure as phophastase 3′ end, can now be employed to restore an —OH group to this 3′ carbon position, thus leaving a terminal nucleotide. Another round of polymerase-mediated incorporation of the next complementary elaborated mono-nucleotide phosphorothiolate compound can then be performed. The next elaborated mono-nucleotide phosphorothiolate compound undergoes polymerase-mediated attachment to the terminal nucleotide, involving conventional nucleophilic attack. Following dye detection, phosphothiolate cleaving, and phosphate removal, the process can thus be repeated successively to determine the sequence of the template.

FIG. 2B shows another embodiment of the present teachings, where a different blocking moiety X is employed. Here, the X blocking moiety is CH₂—(CH₂O)ₙ—NH—(CH₂)ₙ—NH—. Otherwise, this embodiment in FIG. 2B mirrors the embodiment described supra for FIG. 2A.

### Sequencing by Elaborated Di-Nucleotide-Phosphorothiolate Compounds

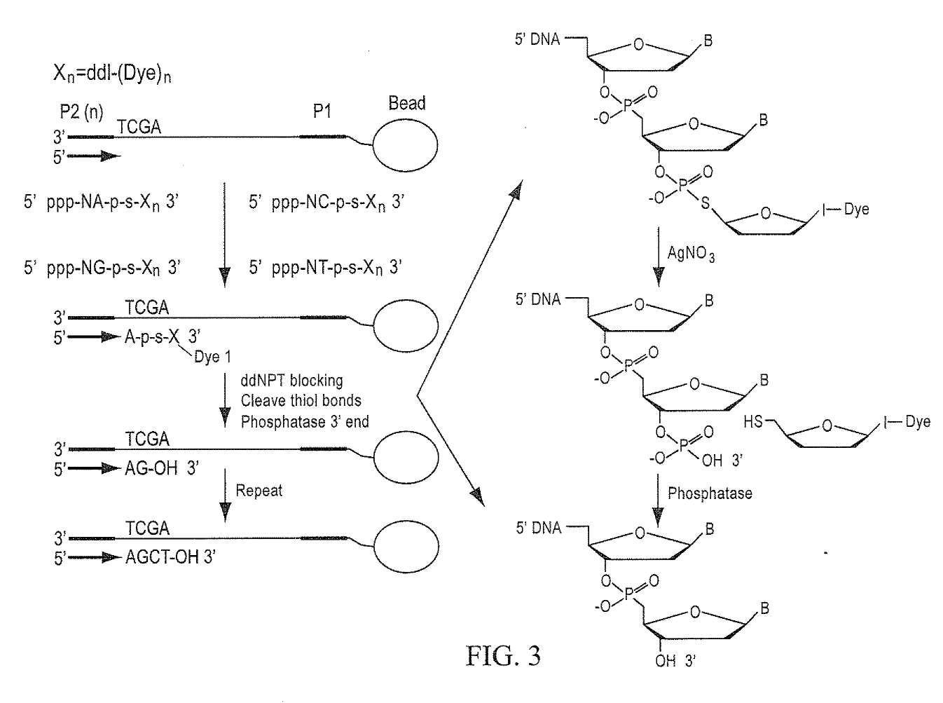

A third aspect of the present teachings is presented in FIGS. 3-6. In FIG. 3, a template polynucleotide is shown attached to a bead. The template can have known ends that can be queried by primers, for example a “P1” end proximal to the bead and a “P2” end distal to the bead. A primer complementary to the P2 end can be hybridized to the template, thus forming a substrate suitable for polymerization with a suitable polymerase. Any suitable polymerase can be employed.

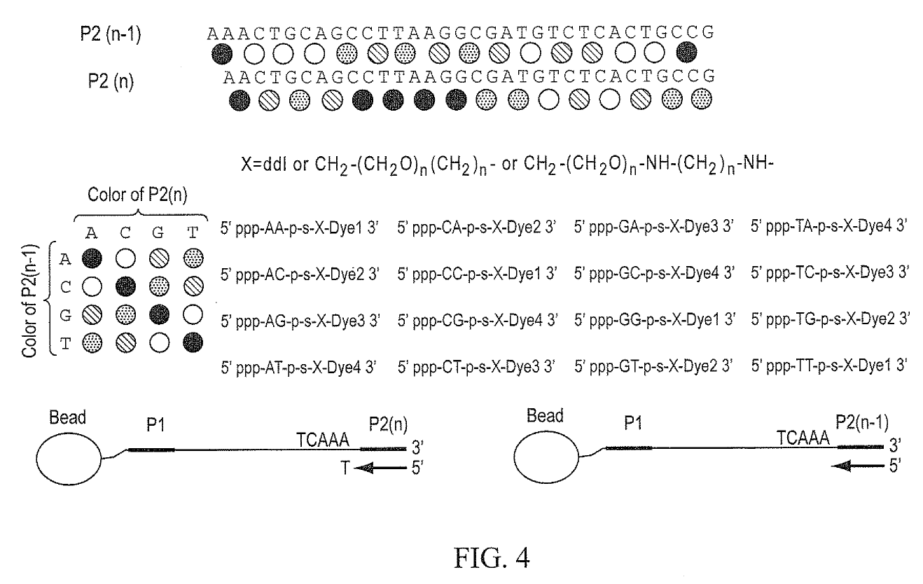

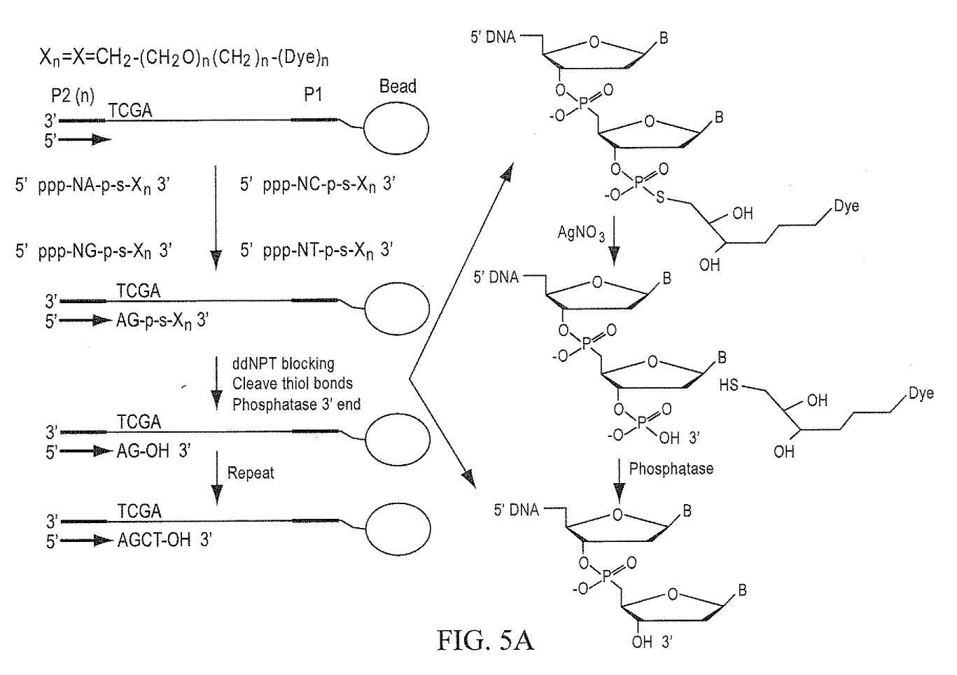

As shown in FIG. 4, a group of all sixteen possible elaborated di-nucleotides can be presented. There can be four families of elaborated di-nucleotides comprising the sixteen elaborated di-nucleotides. Each of the four elaborated di-nucleotides in a family can contain the same label, but vary from each other in the sequence of the two nucleotides. For example, reading diagonally across the 4×4 table of circles on the left side of FIG. 4, the solid fill family contains four members, a 5′AA3′ member, a 5′CC3′ member, a 5′GG3′ member, and a 5′TT3′ member. Each of the four elaborated di-nucleotides in this family can contain a blocking moiety that stops subsequent incorporation of elaborated di-nucleotides. (As was shown in FIG. 3, this blocking moiety can itself be a universal nucleotide (such as inosine, I) in di-deoxy form.) A primer (P2(n)) can be employed. This primer is shown containing a T at its 3′-most position. Extension of this primer results in incorporation of an elaborated di-nucleotide containing TT, the two TT's being complementary to the AA present in the template. Detection of a resulting solid circle Dye 1, indicates to the experimentalist at this point that incorporation of one of the following four elaborated di-nucleotides has occurred:

5′-ppp-AA-p-S-I-Dye-1-3′, or,
5′-ppp-CC-p-S-I-Dye-1-3′, or,
5′-ppp-GG-p-S-I-Dye-1-3′, or,
5′-ppp-TT-p-S-I-Dye-1-3′.

The phosphorothiolate group present between the second nucleotide and the inosine can now be cleaved using a phosphorothiolate cleaving agent, allowing for the removal of the blocking moiety, and its associated Dye. This cleavage will result in a phosphate group on the 3′ carbon of the second nucleotide. Treating with a phosphate removing agent forms a terminal nucleotide bearing an —OH group. Here the second nucleotide of the di-nucleotide is now the terminal nucleotide. Removal of the cleaved label can be achieved with a washing step. A next cycle of incorporation can then be performed and the process can be repeated successively, to form a first “round” of several cycles of incorporation and deprotection, each cycle ultimately adding two nucleotides to the growing extension product. Eventually, the addition of subsequent elaborated di-nucleotides can be stopped, and the resulting extension product stripped from the template.

As shown at the bottom right of FIG. 4, a P2 primer, P2 (n−1), can then be provided that is one nucleotide off-set compared to the first P2 primer (P2(n)), an example of a so-called “offset-primer”. This offset primer lacks the T that was present at the 3′ end of the P2(n) primer. Successive cycles of elaborated di-nucleotide incorporation and deprotection can be repeated. These additions are shifted one nucleotide by the placement of the off-set primer. Determining the sequence of the template can be performed by compilation of the first round of elaborated di-nucleotide incorporation and detection cycles, with the second round of elaborated di-nucleotide incorporation and detection cycles. Such an approach is referred to as “two-base encoding”.

In the present example depicted at the bottom of FIG. 4, one can envision that in the first cycle of the first round, Dye 1 is detected. Detection of Dye 1 tells the experimentalist that one of the following elaborated di-nucleotides was incorporated: 5′AA3′, or 5′CC3′, or 5′GG3′, or 5′TT3′. After the first round is completed, the extension product is stripped from the template. An off-set primer is hybridized to the template, and a first cycle of a second round can be performed. Of note, this offset primer lacks the T at the terminal end that was present in the P2(n) primer employed in the first round. Accordingly, in the depicted embodiment in FIG. 4, Dye 1 would be detected during this first cycle of the second round due to the presence of AA in the template. Based on the signal detected, the experimentalist knows that the elaborated di-nucleotide incorporated in this first cycle of the second round is one of the following; 5′AA3′, or 5′CC3′, or 5′GG3′, or 5′TT3′.

Compiling the results of the first cycle of the first round, with the first cycle of the second round, provides the experimentalist with the information necessary to deduce the identity of the base in the first position of the template: an A. This approach is shown pictorially in the top of FIG. 4, where a given Dye is associated with a given circle (Dye 1 is the solid circle, Dye 2 is the open circle, Dye 3 is the diagonal hashed circle, and Dye 4 is the dotted circle). As a result of these steps, the experimentalist collects an ordered list of probe family names. Here at the bottom of FIG. 4, detection of a solid circle incorporation event in the first cycle of the first round (UT incorporation) using primer P2(n), would eventually be followed by detection of a solid circle incorporation event in the first cycle of the second round (TT incorporation) using off-set primer P2(n−1). Said another way, if the first cycle of the first round yielded a dye associated with a solid circle, then only four possible elaborated di-nucleotides were incorporated during this cycle: 5′AA3′, or 5′CC3′, or 5′GG3′, or 5′TT3′. Since the first cycle of the second round also produced detection of a solid circle, then only four possible elaborated di-nucleotides were incorporated during this cycle: 5′AA3′, or 5′CC3′, or 5′GG3′, or 5′TT3′. Since the experimentalist knows that the off-set primer of the second round hybridized a single nucleotide away from the primer employed in the first round, then necessarily the identity of the first base sequenced of the template is an A. Repeating this process a sufficient number of times allows one to determine the entire sequence of the template. FIGS. 5A and 5B simply indicate that these decoding processes can be employed with various X blocking moieties.

Two-base encoding as applied in a ligation-based sequencing process is described in WO 2006/084132, which is hereby incorporated by reference in its entirety. As employed herein with polymerase-mediate extension of elaborated phosphorothiolate nucleotide compounds, analogous analyses can be performed. For example, it will be appreciated that this two-base encoding, resulting in the ordered list of family names, contains a substantial amount of information, but not in a form that will immediately yield the sequence of interest. Further step(s), at least one of which involves gathering at least one item of additional information about the sequence, must be performed in order to obtain a sequence that is most likely to represent the sequence of interest. The sequence that is most likely to represent the sequence of interest can be referred to as the “correct” sequence, and the process of extracting the correct sequence from the ordered list of probe families is referred to as “decoding”. It will be appreciated that elements in an “ordered list” as described above could be rearranged either during generation of the list or thereafter, provided that the information content, including the correspondence between elements in the list and nucleotides in the template, is retained, and provided that the rearrangement, fragmentation, and/or permutation is appropriately taken into consideration during the decoding process. The ordered list can be decoded using a variety of approaches. Some of theses approaches involve generating a set of at least one candidate sequence from the ordered list of probe family names. The set of candidate sequences may provide sufficient information to achieve an objective. In preferred embodiments one or more additional steps are performed to select the sequence that is most likely to represent the sequence of interest from among the candidate sequences or from a set of sequences with which the candidate sequence is compared. For example, in one approach at least a portion of at least one candidate sequence is compared with at least one other sequence. The correct sequence is selected based on the comparison. In certain embodiments, decoding involves repeating the method and obtaining a second ordered list of probe family names using a collection of probe families that is encoded differently from the original collection of probe families. Information from the second ordered list of probe families is used to determine the correct sequence. In some embodiments information obtained from as little as one cycle of extension, detection, cleavage, and —OH restoration using the alternately encoded collection of probe families is sufficient to allow selection of the correct sequence. In other words, the first probe family identified using the alternately encoded probe family provides sufficient information to determine which candidate sequence is correct.

In some embodiments the elaborated di-nucleotide need not contain a third nucleotide, but can rather comprise a blocking moiety downstream from the second nucleotide. For example, this blocker can be a —CH₂—(CH₂O)ₙ(CH₂)ₙ attached to the 3′ position of the second nucleotide. This approach is depicted in FIG. 5A. An embodiment with a different blocking moiety is depicted in FIG. 5B. Two-base encoding can be performed to sequence the template in much the same fashion as described supra.

In some embodiments, polymerase mediated extension will not be completely efficient. Thus, a capping step can be employed to render un-extendable those nucleic acids that failed to incorporate during the polymerase treatment. For example, following the polymerase treatment, the unincorporated elaborated phosphorothiolates can be removed by washing, and dideoxynucleotides can be added by conventional methods of polymerase extension, such that only those nucleic acids that failed to incorporate earlier will be capped with a dideoxynucleotide. Such capping serves the function of keeping all of the various nucleic acids undergoing sequencing in register. This optional capping step is shown in FIGS. 1-5 with the “ddNTP blocking” language. Various other capping approaches, both reversible and irreversible are known in the art, and can be found described for example in U.S. Pat. No. 6,664,079.

### Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In various embodiments, the present teachings provide a kit for sequencing a template comprising; at least four elaborated nucleotide phosphorothiolate compounds, each elaborated nucleotide phosphorothiolate compound comprising a 3′C—O—PO₂—S—X group, wherein X of the at least four elaborated nucleotide phosphorothiolate compounds comprises a blocking moiety and a distinguishable label; and, a suitable polymerase. In some embodiments, the at least four elaborated nucleotide phosphorothiolate compounds are elaborated mono-nucleotide phosphorothiolate compounds. In some embodiments, the at least four elaborated nucleotide phosphorothiolate compounds are elaborated di-nucleotide phosphorothiolate compounds. In some embodiments, the at least four elaborated nucleotide phosphorothiolate compounds are elaborated oligo-nucleotide phosphorothiolate compounds. In some embodiments, the kit can comprise a phosphate removing agent, such as for example a phosphatase. In some embodiments, the kit can comprise a phosphorothiolate cleaving agent, such as for example AgNO₃. Additional components as taught herein can be included in the kits.

### Certain Exemplary Compositions

In some embodiments, the present teachings provide an elaborated nucleotide phosphorothiolate compound consisting essentially of an elaborated mono-nucleotide, wherein the elaborated mono-nucleotide comprises; a first nucleotide, wherein the 3′ carbon of the first nucleotide is connected to a 3′C—O—PO2-S—X group. In some embodiments, X comprises a blocking moiety selected from the group consisting of a universal nucleotide base, CH₂(CH₂O)n(CH2)m, (CH2)ₐCO—NH—(CH2)_{b}—O—(CH2)_{c}—NH, glycerol, ether, ester, carbohydrate, substituted carbohydrate, carbamate, or phosphoamidite, and wherein X further comprises a label. In some embodiments, n is 0 to 5 and m is 0 to 10 of the CH₂(CH₂O)n(CH2)m compound.

In some embodiments, the present teachings provide an elaborated nucleotide phosphorothiolate compound comprising an elaborated mono-nucleotide, wherein the elaborated mono-nucleotide comprises; a first nucleotide, wherein the 3′ carbon of the first nucleotide is connected to a 3′C—O—PO₂—S—X group.

In some embodiments, the present teachings provide an elaborated nucleotide phosphorothiolate compound consisting of an elaborated mono-nucleotide, wherein the elaborated mono-nucleotide consists of; a first nucleotide, wherein the 3′ carbon of the first nucleotide is connected to a 3′C—O—PO₂—S—X group.

In some embodiments, the present teachings provide an elaborated nucleotide phosphorothiolate compound comprising an elaborated di-nucleotide, wherein the elaborated di-nucleotide comprises; a first nucleotide and a second nucleotide, wherein the 3′ carbon of the second nucleotide comprises a 3′C—O—PO₂—S—X group. In some embodiments, X comprises a blocking moiety selected from the group consisting of a universal nucleotide base, CH₂(CH₂O)n(CH₂)m, (CH2)ₐCO—NH—(CH2)_{b}—O—(CH2)_{c}—NH, glycerol, ether, ester, carbohydrate, substituted carbohydrate, carbamate, or phosphoamidite, and wherein X further comprises a label. In some embodiments, n is 0 to 5 and m is 0 to 5 of the CH₂(CH₂O)ₙ(CH2)m compound.

In some embodiments, the present teachings provide an elaborated nucleotide phosphorothiolate compound consisting essentially of an elaborated di-nucleotide, wherein the elaborated di-nucleotide consists essentially of; a first nucleotide and a second nucleotide, wherein the 3′ carbon of the second nucleotide comprises a 3′C—O—PO₂—S—X group.

In some embodiments, the present teachings provide an elaborated nucleotide phosphorothiolate compound consisting of an elaborated di-nucleotide, wherein the elaborated di-nucleotide consists of; a first nucleotide and a second nucleotide, wherein the 3′ carbon of the second nucleotide comprises a 3′C—O—PO₂—S—X group.

While the present teachings have been described in terms of these exemplary embodiments, the skilled artisan will readily understand that numerous variations and modifications of these exemplary embodiments are possible without undue experimentation. All such variations and modifications are within the scope of the present teachings.

Further, the foregoing description and examples detail certain preferred embodiments of the invention and describes the best mode contemplated by the inventors. It will be appreciated, however, that no matter how detailed the foregoing may appear in text, the present teachings may be practiced in many ways and should be construed in accordance with the appended claims and any equivalents thereof.

We claim:

1. A method of synthesizing a nucleic acid comprising;
hybridizing a primer to a template;
polymerase extending the primer with an elaborated mono-nucleotide phosphorothiolate compound to form an extension product, wherein the elaborated mono-nucleotide phosphorothiolate compound comprises a 3′C—O—PO₂—S—X group;
cleaving the 3′C—O—PO₂—S—X group with a phosphorothiolate cleaving agent, wherein the S—X are removed from the extension product, to leave a 3′PO₄ group;
hydrolyzing the 3′PO₄ group with a phosphate removing agent to leave a 3′OH; and,
repeating (b)-(d) to synthesize the nucleic acid.

2. The method according to claim 1 wherein the elaborated mono-nucleotide phosphorothiolate compound comprises a first nucleotide and a second nucleotide, and wherein the 3′C—O—PO₂—S—X group is attached to the 3′ carbon of the first nucleotide.

3. The method according to claim 1 wherein X of the elaborated mono-nucleotide phosphorothiolate compound is selected from the group consisting of a universal base, CH₂(CH₂O)n(CH2)m, (CH2)ₐCO—NH—(CH2)_{b}—O—(CH2)_{c}—NH, glycerol, an ether group, an ester group, a carbohydrate, a substituted carbohydrate, a carbamate, or a phosphoamidite.

4. The method according to claim 3 wherein n is 0 to 5 and m is 0 to 10.

5. The method according to claim 1 wherein the phosphorothiolate cleaving agent is a metal compound selected from the group consisting of Au, Ag, Hg, Cu, Mn, Zn, or Cd, or is a halide compound selected from the group consisting of iodine or bromine.

6. The method according to claim 1 wherein the phosphate removing agent is selected from the group consisting of a phosphatase and a reversible kinase.

7. A method of sequencing a nucleic acid comprising;
hybridizing a primer to a template;
polymerase extending the primer with an elaborated nucleotide phosphorothiolate compound to form an extension product, wherein the elaborated nucleotide phosphorothiolate compound comprises a 3′C—O—PO₂—S—X, wherein X further comprises a label;
detecting the label to determine base identity or to determine probe family identity;
cleaving the 3′C—O—PO₂—S—X group with a phosphorothiolate cleaving agent, wherein the S—X are removed from the elongated strand, to leave a 3′PO₄ group on a terminal nucleotide;
cleaving the 3′PO₄ with a phosphate removing agent to leave a 3′OH on the terminal nucleotide; and
repeating (b)-(e) to sequence the nucleic acid.

8. The method according to claim 7 wherein the elaborated nucleotide phosphorothiolate compound comprises an elaborated di-nucleotide, wherein the elaborated di-nucleotide comprises a first nucleotide and a second nucleotide, and wherein the 3′C—O—PO₂—S—X group is attached to the 3′ carbon of the second nucleotide.

9. The method according to claim 8 wherein X is a nucleotide containing a universal base, wherein the universal base comprises a blocking moiety at its 3′ carbon.

10. The method according to claim 9 wherein the universal base of the nucleotide comprises the label, and wherein the label is attached to either the universal base or to the blocking moiety at the 3′ carbon of the universal base.

11. The method according to claim 7 wherein X is selected from the group consisting of CH₂(CH₂O)n(CH2)m, (CH2)ₐCO—NH—(CH2)_{b}—O—(CH2)_{c}—NH, glycerol, ether, ester, carbohydrate, substituted carbohydrate, carbamate, or phosphoamidite.

12. The method according to claim 11 wherein n is 0 to 5 and wherein m is 0 to 10.

13. The method according to claim 7 wherein the elaborated nucleotide phosphorothiolate compound comprises an elaborated mono-nucleotide, wherein the elaborated mono-nucleotide comprises a first nucleotide, and wherein the 3′C—O—PO₂—S—X group is attached to the 3′ carbon of the first nucleotide.

14. The method according to claim 13 wherein X is a nucleotide containing a universal base, wherein the universal base comprises a blocking moiety at its 3′ carbon.

15. The method according to claim 14 wherein the universal base of the nucleotide comprises the label, and wherein the label is attached to either the base or to the blocking moiety at the 3′ carbon.

16. The method according to claim 7 wherein the phosphorothiolate cleaving agent is a metal compound selected from the group consisting of Au, Ag, Hg, Cu, Mn, Zn, or Cd, or is a halide compound selected from the group consisting of iodine or bromine.

17. The method according to claim 7 wherein the phosphate removing agent is selected from the group consisting of a phosphatase and a reversible kinase.

18. An elaborated nucleotide phosphorothiolate compound consisting essentially of an elaborated mono-nucleotide, wherein the elaborated mono-nucleotide comprises; a first nucleotide, wherein the 3′ carbon of the first nucleotide is connected to a 3′C—O—PO₂—S—X group.

19. The compound according to claim 18 wherein X comprises a blocking moiety selected from the group consisting of a universal nucleotide base, CH₂(CH₂O)n(CH2)m, (CH2)ₐCO—NH—(CH2)_{b}—O—(CH2)_{c}—NH, glycerol, ether, ester, carbohydrate, substituted carbohydrate, carbamate, or phosphoamidite, and wherein X further comprises a label.

20. The composition according to claim 19 wherein n is 0 to 5 and m is 0 to 10.

21. An elaborated nucleotide phosphorothiolate compound comprising an elaborated di-nucleotide, wherein the elaborated di-nucleotide comprises;
a first nucleotide and a second nucleotide, wherein the 3′ carbon of the second nucleotide comprises a 3′C—O—PO₂—S—X group.

22. The compound according to claim 21 wherein X comprises a blocking moiety selected from the group consisting of a universal nucleotide base, CH₂(CH₂O)n(CH2)m, (CH2)ₐCO—NH—(CH2)_{b}—O—(CH2)_{c}—NH, glycerol, ether, ester, carbohydrate, substituted carbohydrate, carbamate, or phosphoamidite, and wherein X further comprises a label.

23. The composition according to claim 22 wherein n is 0 to 5 and m is 0 to 5.

24. A kit for sequencing a template comprising;
(b) at least four elaborated nucleotide phosphorothiolate compounds, each elaborated nucleotide phosphorothiolate compound comprising a 3′C—O—PO₂—S—X group, wherein X of the at least four elaborated nucleotide phosphorothiolate compounds comprises a blocking moiety and a distinguishable label; and,
(c) a suitable polymerase.

25. The kit according to claim 24 wherein the at least four elaborated nucleotide phosphorothiolate compounds are elaborated mono-nucleotide phosphorothiolate compounds.
